(19) United States
(12) Patent Application Publication
(10) Pub. No.: US 2015/0157785 A1
(43) Pub. Date: Jun. 11, 2015

(54) DEVICES AND KITS USED IN HOLDING CYLINDRICAL OBJECTS

(71) Applicant: James G. Hilliard, Portland, OR (US)

(72) Inventor: James G. Hilliard, Portland, OR (US)

(73) Assignee: OREGON HEALTH & SCIENCE UNIVERSITY, PORTLAND, OR (US)

(21) Appl. No.: 14/564,557

(22) Filed: Dec. 9, 2014

Related U.S. Application Data
(60) Provisional application No. 61/913,996, filed on Dec. 10, 2013.

(51) Int. Cl. A61M 5/14; F16L 3/02; A61M 39/22

(52) U.S. Cl. CPC A61M 5/1415; A61M 39/22; F16L 3/02; A61M 2039/229

(57) ABSTRACT

Devices for use in holding cylindrical objects are disclosed. The device includes a U-shaped portion that cradles the cylindrical object. The U-shaped portion comprises two arms. A straight portion is joined to one of the arms. The straight portion includes a hole traversing the length of the straight portion. The hole is configured to hold a fastener such as a screw or bolt. The fastener is in turn configured to engage a hole in an attachment mechanism.

### FIELD

Generally, the field is holders for cylindrical objects such as rods and tubes. More specifically, the field is holders for rigid tubes used as ports or valves for intravenous tubing such as stopcocks.

### BACKGROUND

Management and organization of intravenous (IV) tubing in clinically ill patients is challenging. Often, IV tubing and ports such as stopcocks will fall or be displaced and are thereby exposed to contamination. Often, the tubing and ports are allowed to hang free or at best, secured to a solid surface with tape. Clearly a reliable, secure, inexpensive holder for cylindrical objects, including rigid cylindrical objects that serve as components in systems that deliver intravenous fluids to patients, such as stopcocks is needed.

### SUMMARY

Disclosed herein is a device that may be used to hold a cylindrical object in place. The device includes a U-shaped portion configured to cradle the object. It also includes a straight portion joined to one of the arms of the U-shaped portion. The straight portion includes a hole traversing the length of the straight portion. The hole is configured to accept a fastener that in turn is configured to engage a hole in an attachment mechanism.

It is an object of the invention to provide a device that stably holds a component of a system that provides intravenous fluids to a patient (such as a stopcock) in place.

It is an object of the invention to provide a device that stably holds a piece of laboratory glassware or plasticware (such as a pipet or burette) in place.

It is an object of the invention to provide a device that is easily sterilized.

It is an object of the invention to provide a device that can be disposable.

It is an object of the invention to provide a device that can be attached to a wide variety of solid substrates or surfaces.

### BRIEF DESCRIPTION OF THE DRAWINGS

FIG. 1 is a cross-section of one example of the device.

FIG. 2 is a top view of one example of the device.

FIG. 3 is a perspective view of the device including additional components that interact with the device.

FIG. 4 is a cross-section of an example of the device with a straight portion integrated into the arm of the U-shaped portion.

FIG. 5 is a cross-section of an example of the device with a rounded bottom to the U-shaped portion.

### DETAILED DESCRIPTION

Disclosed herein is a device that can be used to cradle a cylindrical object such as a hard plastic or metal tube in place. The device comprises a U-shaped portion that is configured to cradle the cylindrical object, a straight portion that is joined to one of the arms of the U-shaped portion, and a hole traversing the length of the straight portion. The hole in the straight portion is configured to accept a fastener. The fastener in turn is configured to engage a second hole in an attachment mechanism, which is then gripped to a stable substrate.

In general, the U-shaped portion comprises a rounded area and two arms that extend from each end of the rounded area in a shape reminiscent of the Roman letter ‘U’. The size and shape of the U-shaped portion is configured to cradle the cylindrical object such that at least part of the U-shaped portion is in contact with the cylindrical object through at least part of its circumference. In some examples, the cylindrical object tightly snaps into the U shaped portion.

The arms may be any length, including no length beyond the rounded area. In some examples, the ends of the arms curve or angle towards one another in order to more tightly secure the cylindrical object. Therefore, in some examples, the U-shaped portion is in contact with the full circumference of the cylindrical object. In other examples, the arms extend vertically from the ends of the rounded area. The bottom of the U-shaped portion may be in any configuration including substantially parallel to and following the shape of the top of the U-shaped portion to form a rounded bottom or forming right angles with the arms of the U-shaped portion. The U-shaped portion is configured to firmly the engage cylindrical object and may be configured to snap into and out of the U-shaped portion. One of skill in the art in light of this disclosure could construct a U-shaped portion to fit any size of cylindrical object. In some examples, the U-shaped portion comprises a writing surface.

The straight portion is joined to one of the arms of the U-shaped portion such that the length of the U-shaped portion makes substantial contact with the length of the straight portion. The straight portion may be longer than, shorter than, or the same length as the arm of the U-shaped portion to which it is joined. In some examples, the straight portion and the U-shaped portion are molded or machined out of a single piece of material. In other examples, the straight portion and the U-shaped portion are provided as separate components that are fused together using, for example, an adhesive. The straight portion may be of any width and thickness relative to the arm of the U-shaped portion. It may be wider, narrower, thicker, or thinner, up to and including being fully integrated into one of the arms of the U-shaped portion.

The hole in the straight portion traverses the entire length of the straight portion. The hole can be in any position relative to the center of the straight portion, including in the center, substantially in the center, or in any direction off-center depending on the dimensions of the device. The hole can be threaded in order to engage threads on a fastener comprising a screw or bolt. As disclosed herein, the length of a portion is equivalent to the longest dimension of the portion. Width and thickness may be defined in relation to the length.

The device can be composed of any appropriate material including soft or hard plastic, natural or synthetic rubber, or metal. The device may be solid or hollow. One of skill in the art in light of this disclosure would be able to select a proper material out of which to make the device.

Any appropriate fastener can be placed into the hole, including a screw, a bolt (including a nut or not), a brad, a nail, or any other metal or plastic rod that can be passed through the hole and engage an attachment mechanism on the bottom of the straight portion.

The attachment mechanism can be any device that can be configured to engage the fastener and grip a solid substrate. The attachment mechanism may be an alligator clip, clothespin, binder clip, or other similar clamp comprising jaws that are held closed in a resting position by a spring or other similar device and opened when pressure is applied to tabs at the opposite end from the jaws. The attachment mechanism can also include such devices as a paper clip, plastic clip or other clip that slides over the end of a piece of paper or fabric and is held in place by friction. Such attachment mechanisms can also involve snapping together the ends of the clip to hold it in place. In other examples, the attachment mechanism can comprise a hook and loop closure such as Velcro® wherein, for example, the hook fabric is attached to the device and the loop fabric is attached to a the solid substrate. The attachment mechanism can also be a suction cup or other device that adheres temporarily to a surface. One of skill in the art in light of this disclosure would be able to select and/or modify any known or yet to be disclosed attachment mechanism that may be used to secure the device to a particular solid substrate.

In particular examples the attachment mechanism comprises hemostatic forceps. Such a device is in the shape of a pair of scissors with two pieces attached at a pivot joint. One end generally comprises finger holes that facilitate opening and closing of the jaws at the other end of the device about the pivot joint. The forceps can have gripping pads or any other configuration that will securely attach the forceps to the solid substrate at the jaws. The forceps can also comprise a locking mechanism near the finger holes that holds the jaws closed when locked.

The solid substrate can be any surface or object on and/or secured to a surface that can engage the attachment mechanism. Examples in the hospital setting include an IV pole, a tray secured to an IV pole, a surgical drape, a surgical table, a sheet, the patient's clothing, a bed, or any other appropriate object. In a research setting, the solid substrate can be a lab bench, a laboratory stand, a ring stand, a backsplash behind a lab bench, a wall, a window, or any other desirable substrate.

In particular examples, the device is a component of a kit. A kit is an assemblage of components used to perform a particular task, such as a kit used to set up intravenous delivery of fluids to a patient. Use of kits provides advantages to the end user of the method in that the components can be standardized, subjected to quality control and assurance, subject to sterilization, or optimized for maximal efficacy. Additionally, a kit provides the advantage that the components of the kit are obtained from a single source. The components of a kit are generally enclosed in one or more containers appropriate for storage such as bags or sealed pouches. The containers may be further packaged into a secondary container such as boxes, bags, or any other enclosure. Kits as disclosed herein can be assemblages comprising the device, a fastener, and an attachment mechanism as described herein. They may also comprise the cylindrical device that the device is configured to hold (such as a stopcock or other component of a system used in the delivery of intravenous fluids). In still further examples, the attachment mechanism is the set of hemostatic forceps described above.

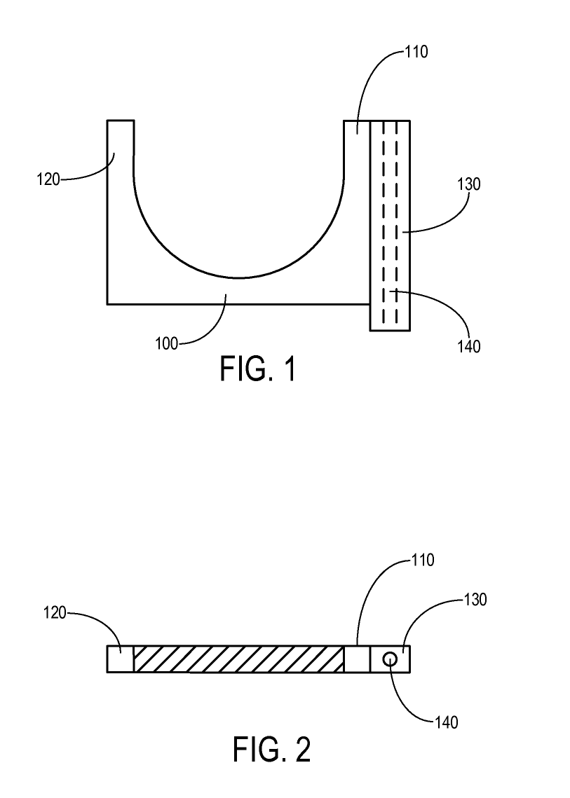

Referring now to FIG. 1, which is a cross section of the device: The device comprises a U-shaped portion 100 with a first arm 110 and a second arm 120. A straight portion 130 is joined with a first arm 110 of the U-shaped portion 100. A hole 140 traverses the length of the straight portion.

Referring now to FIG. 2 which is a top view of the device: The U-shaped portion 100, the first arm 110 and the second arm 120 are indicated. The straight portion 130 is joined with the first arm. In this example, the hole is approximately in the center of the straight portion.

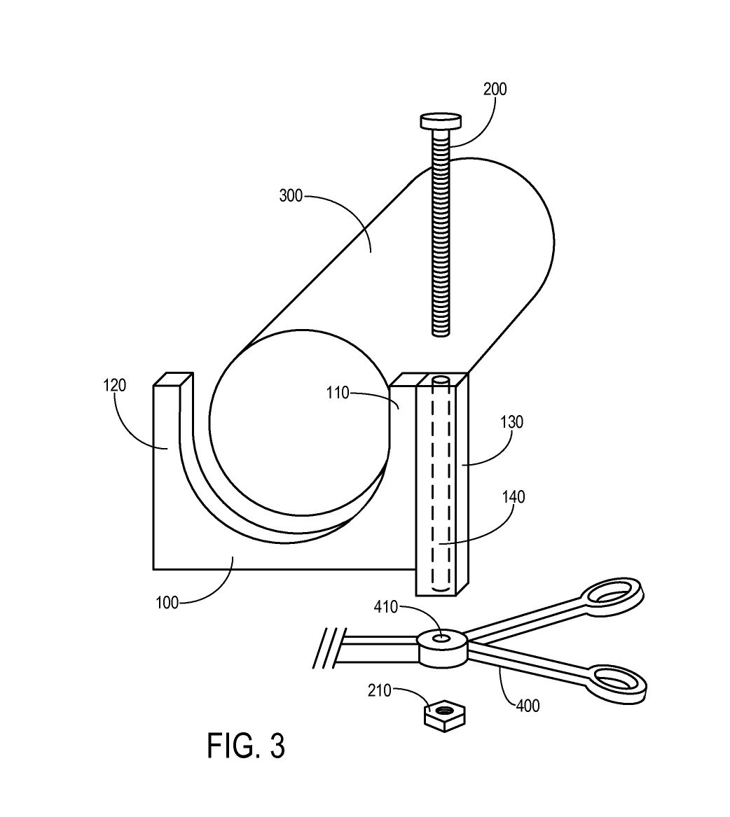

Referring now to FIG. 3 which is a perspective view of the device along with additional items that interact with the device: The device comprises a U-shaped portion 100 with a first arm 110 and a second arm 120. A straight portion 130 is joined to the first arm. The straight portion comprises a hole 140 traversing the length of the straight portion. The U-shaped portion is configured to engage a cylindrical object 300. The hole is configured to accept a fastener 200, exemplified by a threaded bolt in this figure. The fastener engages an attachment mechanism 400, through a second hole 410. In this figure, the attachment mechanism is exemplified by hemostatic forceps. In the example given in the figure, when the second hole 410 is engaged by the fastener 200, the second hole forms a pivot joint in the hemostatic forceps. In the example given in the figure, a nut 210 is used to secure the forceps to the device.

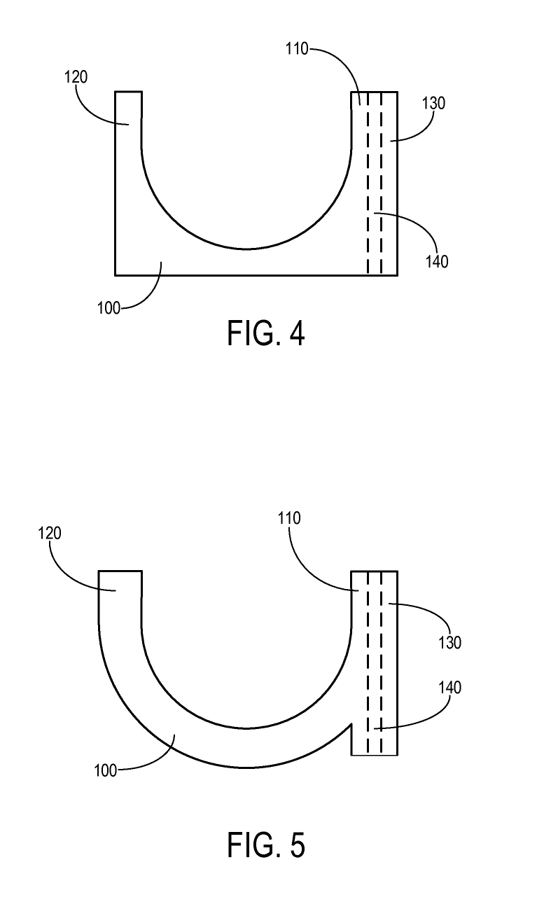

Referring now to FIG. 4 which is a cross section of an alternative form of the device wherein the straight portion 130 is integrated into, or alternatively formed by the first arm 110.

Referring now to FIG. 5 which is a cross section of an alternative form of the device wherein the bottom of the U-shaped portion is substantially parallel to and follows the shape of the top of the U-shaped portion to form a rounded bottom.

1. A device used to hold a cylindrical object, the device comprising:
a U-shaped portion configured to cradle the object;
a straight portion joined to a first arm of the U-shaped portion; and
a first hole traversing the length of the straight portion, said hole configured to accept a fastener, said fastener configured to engage a second hole in an attachment mechanism.

2. The device of claim 1 wherein the U-shaped portion and the straight portion are provided as separate units that are fused together.

3. The device of claim 2 wherein the U-shaped portion and the straight portion are fused using an adhesive.

4. The device of claim 1 wherein the U-shaped portion and the straight portion are provided as a single piece of material.

5. The device of claim 1 wherein the top of the first arm of the U-shaped portion is flush with the top of the straight portion.

6. The device of claim 1 wherein the bottom of the first arm of the U-shaped portion is flush with the bottom of the straight portion.

7. The device of claim 1 wherein the fastener comprises a screw or bolt.

8. The device of claim 7 wherein the first hole is threaded to engage the fastener.

9. The device of claim 7 wherein the fastener is configured to engage a thread in the second hole.

10. The device of claim 1 wherein the attachment mechanism comprises locking forceps.

11. The device of claim 10 wherein the second hole serves as the pivot joint in the locking forceps when the locking forceps are engaged with the fastener.

12. A kit comprising:
the device of claim 1;
a fastener; and
an attachment mechanism.

13. The kit of claim 12 further comprising a cylindrical object configured that the U-shaped portion of the device is configured to fit.

14. The kit of claim 12 wherein the cylindrical object is a component of a system used in the delivery of intravenous fluids.

15. The kit of claim 12 wherein the attachment mechanism comprises locking forceps.

16. The kit of claim 15 wherein the fastener comprises a bolt configured to engage a hole in the locking forceps wherein the hole serves as the pivot joint when the bolt is engaged with the hole.

17. The kit of claim 16 further comprising a nut configured to engage the bolt.
